(19) United States
(12) Patent Application Publication
(10) Pub. No.: US 2002/0114780 A1
(43) Pub. Date: Aug. 22, 2002

(54) Methods of increasing distribution of therapeutic agents

(76) Inventors: Krys Bankiewicz, Piedmont, CA (US); John Hamilton, Washington, DC (US); Edward Oldfield, Philomont, VA (US); Heidi Phillips, Palo Alto, CA (US)

Correspondence Address: NEEDLE & ROSENBERG, P.C., The Candler Building, Suite 1200, 127 Peachtree Street, N.E., Atlanta, GA, 30303-1811, US

(21) Appl. No.: 09/999,203

(22) Filed: Nov. 30, 2001

Related U.S. Application Data
(60) Provisional application No. 60/250,286, filed on Nov. 30, 2000.

(51) Int. Cl. A61K 38/19; A61K 31/727

(52) U.S. Cl. 424/85.1; 514/56

(57) ABSTRACT

The present invention provides a method of increasing the volume of distribution of a therapeutic agent in a tissue in a subject during localized delivery comprising administering to the tissue in the subject a therapeutic agent and a facilitating agent, whereby the inclusion of the facilitating agent increases the volume of distribution of the therapeutic agent in the tissue. The invention also provides a method of increasing the pharmacological activity of a therapeutic agent in a tissue in a subject during localized delivery, comprising administering to the tissue in the subject a therapeutic agent and a facilitating agent, whereby the inclusion of the facilitating agent increases the pharmacological activity of the therapeutic agent in the tissue.

This application claims the benefit of priority of U.S. Provisional Application No. 60/250,286, filed Nov. 30, 2000. The 60/250,286 provisional patent application is herein incorporated by this reference in its entirety.

### FIELD OF THE INVENTION

This invention relates to methods for increasing the volume of distribution and/or the pharmacological activity of a therapeutic agent using various modes of localized delivery.

### BACKGROUND OF THE INVENTION

A variety of localized delivery methods are available, such as convection enhanced delivery (CED) which provides for the distribution of therapeutic agents in a homogeneous, targeted fashion to solid tissues in clinically useful volumes. However, the binding of therapeutic agents to binding sites other than the intended target of the therapeutic agents limits the volume of distribution (V_{d}).

Within the transforming growth factor-β superfamily of signaling molecules, a subfamily of trophic factors with homology to GDNF has been identified. This subfamily of ligands consists of GDNF (18), Neurturin (NTN) (16), Artemin (ART) (2) and Persephin (PSP) (19). Trophic factors homologous with GDNF, except Persephin, have receptors which are expressed in the mammalian CNS. Members of the GDNF family support the survival of dopaminergic neurons in the substantia nigra, spinal and facial motor neurons in in vitro survival and in vivo injury models, indicating that they may have utility in the treatment of neurodegenerative disorders (26). NTN and GDNF increase high affinity dopamine uptake and utilization in vitro (18) and in vivo (13). Other trophic factors implicated in the etiology and possible treatment of neurological disorders are nerve growth factor for Alzheimer's disease (20, 29) and ciliary neurotrophic factor for Huntington's disease (7). However, their potential efficacy as therapeutic agents may be limited by the ability to deliver them at an effective concentration over clinically significant volumes.

To overcome this limitation, the present invention provides methods of increasing the volume of distribution of a therapeutic agent during various modes of delivery, such as, for example, CED, comprising administering a therapeutic agent and a facilitating agent to, for example, a tissue or a subject, whereby the inclusion of the facilitating agent increases the volume of distribution of the therapeutic agent. These methods can be utilized to treat a variety of disorders, such as neurodegenerative disorders and cancer.

### SUMMARY OF THE INVENTION

In one embodiment, the present invention provides a method of increasing the volume of distribution of a therapeutic agent in a tissue in a subject during localized delivery (e.g., CED, intracerebral injection, intraventicular injection) comprising administering to the tissue in the subject a therapeutic agent and a facilitating agent, whereby the inclusion of the facilitating agent increases the volume of distribution of the therapeutic agent in the tissue.

Further provided by the present invention is a method of increasing the pharmacological activity of a therapeutic agent in a tissue in a subject during localized delivery (e.g., CED, intracerebral injection, intraventricular injection) comprising administering to the tissue in the subject a therapeutic agent and a facilitating agent, whereby the inclusion of the facilitating agent increases the pharmacological activity of the therapeutic agent in the tissue.

Also provided by the present invention is a method of treating a neurodegenerative disorder in a subject in need of such treatment, comprising administering to the subject a therapeutic agent and a facilitating agent, wherein the therapeutic agent and the facilitating agent are administered via localized delivery (e.g., CED, intracerebral delivery, intraventricular delivery).

The present invention further provides a method of increasing dopamine metabolism in a tissue in a subject during localized delivery of a therapeutic agent (e.g., by CED, intracerebral delivery, intraventricular delivery) comprising administering to the tissue in the subject a therapeutic agent and a facilitating agent, whereby the inclusion of the facilitating agent increases dopamine metabolism in the tissue.

### BRIEF DESCRIPTION OF THE DRAWINGS

FIG. 1a shows CED of trophic factors of the GDNF family with and without heparin. 5 μg of trophic factor in 5 μl of infusate infused at 0.2 μl/min. Animals were sacrificed immediately. Immunohistochemical staining was performed on 40 μm sections with antibodies to the respective trophic factor in the GDNF ligand family. Scale bar=1 mm.

FIG. 1b illustrates CED of BSA with and without heparin. 5 μg BSA in 5 μl of vehicle infused at 0.2 μl/min. Animals were sacrificed immediately following infusion. Immunohistochemical staining was performed on 40 μm sections with antibodies to BSA. Scale bar=1 mm.

FIG. 2a shows the effect of co-infusion with heparin on distribution of BSA and the GDNF family of trophic factors in rat striatum. The V_{d} of each protein infused alone (BSA n=6, GDNF n=4, NTN n=4, ART n=5) was compared to co-infusion with heparin (GDNF n=4, NTN n=4, ART n=4). Values are expressed as the mean +/−S.D. Unpaired Student t-test indicates significance at P<0.05 (*); (**) P<0.0005.

FIG. 2b shows the effect of co-infusion with heparin on the V_{d}/Vₗ ratio of the GDNF family of trophic factors. The V_{d} of each protein infused alone (BSA n=6, GDNF n=4, NTN n=4, ART n=5) was compared to co-infusion with heparin (GDNF n=4, NTN n=4, ART n=4). Values are mean +/−S.D. (*) P<0.005; (**) P<0.0005.

FIG. 3 illustrates high volume NTN intrastriatal infusion. 50 μg of NTN in 20 μl of vehicle was infused at 0.2 μl/min into the right striatum. Animals were sacrificed immediately. Immunohistochemical staining was performed on 40 μm sections with antibodies to the NTN.

FIG. 4 shows that heparin co-infusion with NTN enhances upregulation of dopamine utilization by NTN. Animals in each group received a right striatal infusion of 5 μl vehicle with heparin (n=5), 5 μg of NTN alone in 5 μl of vehicle (n=7), or 5 μg NTN in 5 μl vehicle with heparin (n=4). Animals were sacrificed at 4 days. DOPAC/DA ratios were measured by HPLC. The DOPAC/DA ratio in each subject is expressed as a percentage of the uninjected hemisphere. The Fisher test was used. Results are indicated as the ratio of the mean +/−S.E. The DOPAC/DA ratio was significantly increased in both the NTN group and the NTN+heparin group when compared to the vehicle+heparin group (*); (P=0.0179 and <0.0001, respectively). Furthermore, the NTN+heparin group had a significantly increased DOPAC/DA ratio when compared to the group which received NTN alone (+) (P=0.0004).

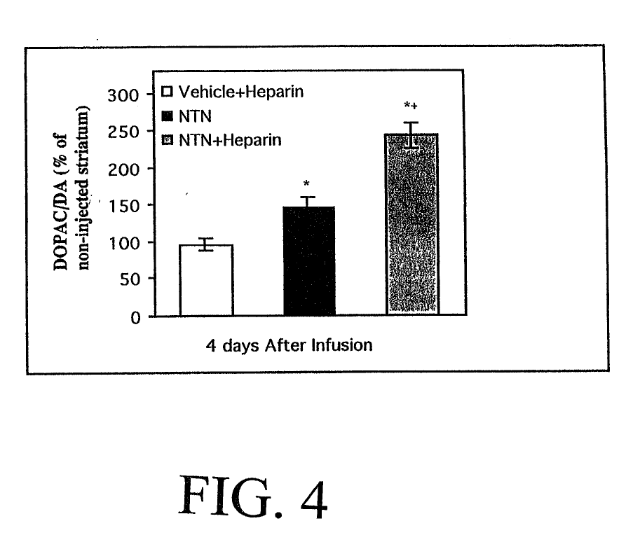

### DETAILED DESCRIPTION OF THE INVENTION

The present invention may be understood more readily by reference to the following detailed description of the preferred embodiments of the invention and the Example included herein.

Before the present methods are disclosed and described, it is to be understood that this invention is not limited to specific proteins or specific methods. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise.

The present invention is based on the surprising and unexpected discovery that the volume of distribution and/or pharmacological activity of a therapeutic agent can be increased during localized delivery. The administration of therapeutic agents of the present invention can be via any localized delivery system that allows for the enhancement of or an increase in the pharmacological activity of a therapeutic agent when a facilitating agent is included in the delivery. Examples of such delivery systems include, but are not limited to CED, intraventricular delivery, intracerebroventricular delivery and intracerebral delivery. Examples of other delivery systems include localized injection via hypodermic needle or an injection gun.

As used herein, localized delivery is defined as delivery of a therapeutic agent into a region of the body such as an organ, or a part of an organ. Examples of organs for which localized delivery is suitable include but are not limited to, heart, kidney, liver, brain and lung. Without limiting the invention to this particular example, the brain is an organ that is composed of specific regions or parts defined by either anatomical or physiological function and localized delivery can be to one or more of the specific regions or parts.

Thus, in one embodiment, the therapeutic agents and facilitating agents of the present invention can be administered via CED. CED is well established in the art and the skilled artisan would know how to adapt CED protocols in order to deliver a particular combination of therapeutic agent and facilitating agent to a solid tissue. U.S. Pat. No. 5,720,720 describes CED and is hereby incorporated by reference in its entirety.

The present invention provides a method of increasing the volume of distribution of a therapeutic agent in a tissue in a subject during localized delivery, such as convection enhanced delivery, localized injection, intraventricular delivery and/or intracerebral delivery, comprising administering to the tissue in the subject a therapeutic agent and a facilitating agent, whereby the inclusion of the facilitating agent increases the volume of distribution of the therapeutic agent in the tissue.

By “increasing the volume of distribution of a therapeutic agent” is meant that the volume of distribution of a therapeutic agent when administered with a facilitating agent is greater than the volume of distribution observed or detected when the therapeutic agent is administered in the absence of a facilitating agent. The volume of distribution can be measured as described in the Examples and by methods known in the art. For example, neuroimaging can be used for in vivo detection. Such neuroimaging methods are known in the art and include magnetic resonance imaging (MRI), positron emission topography (PET), single photon emission computed tomography (SPECT) and computed tomography (CT) scan.

In the methods of the present invention, the facilitating agent can be administered prior to administration of the therapeutic agent, after administration of the therapeutic agent and/or simultaneously with the administration of the therapeutic agent. Furthermore, one or more facilitating agents can be administered with one or more therapeutic agents.

The present invention also provides a method of increasing the pharmacological activity of a therapeutic agent in a tissue in a subject during localized delivery, such as CED, localized injection, intraventricular delivery and/or intracerebral delivery, comprising administering to the tissue in the subject a therapeutic agent and a facilitating agent, whereby the inclusion of the facilitating agent increases the pharmacological activity of the therapeutic agent in the tissue. Pharmacological activity can be measured as described in the Examples and by other methods known in the art.

As used herein, the term “pharmacological activity” refers to the inherent physical properties of a therapeutic agent. These properties include, but are not limited to, binding properties, half-life, stability, ability to effect signal transduction and other pharmacokinetic properties which would be known to one skilled in the art.

The therapeutic agents of the present invention can include, but are not limited to, proteins, drugs, antibodies, antibody fragments, immunotoxins, chemical compounds, protein fragments and toxins. For example, the therapeutic agent of the present invention can be a GDNF ligand, such as GDNF, NTN or artemin.

The facilitating agent, as used herein, can be any agent that increases the volume of distribution and/or pharmacological activity of a therapeutic agent. The facilitating agents of the present invention can include, but are not limited to, proteins, drugs, antibodies, antibody fragments, chemical compounds, toxins or protein fragments.

For example, if it is desirable to administer a therapeutic agent to the brain and the skilled artisan knows or has determined that, in addition to interacting with its target binding site, a given therapeutic agent interacts with an alternate binding site, a facilitating agent would be administered either before, after and/or simultaneously with the administration of the therapeutic agent. The facilitating agent would interact with the alternate binding site in order to prevent binding of the therapeutic agent to this alternate binding site, and thus allow the therapeutic agent to interact preferentially with the target binding site thereby resulting in increased volume of distribution and/or pharmacological activity of the therapeutic agent.

Similarly, if one skilled in the art knows or has determined that this therapeutic agent interacts with a target binding site as well as with a binding protein, a facilitating agent, such as an antibody to the binding protein, would be administered either before, after and/or simultaneously with the administration of the therapeutic agent in order to prevent the therapeutic agent from interacting with the binding protein, and thus allow the therapeutic agent to interact preferentially with the target binding site thereby resulting in increased volume of distribution and/or pharmacological activity of the therapeutic agent. Because there are numerous types of protein-protein interactions that can be disrupted by a facilitating agent in order to increase distribution and/or pharmacological activity of a therapeutic agent, the above-mentioned examples are only exemplary and are not meant to limit the present invention in any way.

One skilled in the art could determine the appropriate combination of a therapeutic agent and a facilitating agent. For example, as described in the Examples herein, heparin can be used as a facilitating agent for the delivery of a therapeutic agent, such as a GDNF ligand (e.g., GDNF, NTN or artemin). Agents that mimic heparin can also be utilized as facilitating agents to deliver a GDNF ligand or other therapeutic agents that interact with heparin receptors. Therefore, once a particular facilitating agent is identified, other drugs, compounds, proteins, antibodies etc., that mimic that particular facilitating agent can be utilized in the methods of the present invention.

Examples of other therapeutic agents that can be employed in the methods of this invention wherein heparin is a facilitating agent include, but are not limited to, GDNF family ligands, PDGF (platelet-derived growth factor) family ligands, FGF (fibroblast growth factor) family ligands, VEGF (vascular endothelial growth factor) and its homologs, HGF (hepatocyte growth factor), midkine, pleiotrophin, amphiregulin, platelet factor 4, CTGF, Interleukin 8, gamma interferon, members of the TGF-beta family, Wnt family ligands, WISP family ligands (Wnt-induced secreted proteins), thrombospondin, TRAP (thrombospondin-related anonymous protein), RANTES, properdin, F-spondin, DPP (decapentaplegic) and members of the Hedgehog family.

Examples of therapeutic agents which can be employed in the methods of this invention wherein the facilitating agent is an antibody directed against the heparin-binding domain of the therapeutic agent can include, but are not limited to, GDNF family ligands, PDGF (platelet-derived growth factor) family ligands, FGF (fibroblast growth factor) family ligands, VEGF (vascular endothelial growth factor) and its homologs, HGF (hepatocyte growth factor), midkine, pleiotrophin, amphiregulin, platelet factor 4, CTGF, Interleukin 8, gamma interferon, members of the TGF-beta family, Wnt family ligands, WISP family ligands (Wnt-induced secreted proteins), thrombospondin, TRAP (thrombospondin-related anonymous protein), RANTES, properdin, F-spondin, DPP (decapentaplegic) and members of the Hedgehog family.

Other examples of pairs of therapeutic agents and facilitating agents which can be used in the methods of the invention include, but are not limited to, GDNF and GFRα1, GDNF and an antibody to GDNF, neurturin and GFRα1, neurturin and GFRα2, neurturin and an antibody to neurturin, artemin and GFRα23, artemin and antibody to artemin, persephin and GFRα4, persephin and an antibody to persephin, NGF and TrkA-Ig, NGF and an antibody to NGF, BDNF and TrkB-Ig, BDNF and TrkC-Ig, BDNF and an antibody to NGF, NT3 and TrkC-Ig, NT3 and an antibody to NT3, IGF-1 and IGF-BP1, IGF-1 and IGF-BP-2, IGF-1 and IGF-BP3, IGF-1 and an antibody to IGF-1, sonic hedgehog and sonic patched, sonic hedgehog and an antibody to sonic hedgehog.

The methods of the present invention can be utilized to deliver therapeutic agents and facilitating agents to tissues such as the brain, heart, lung, solid tumors, liver, kidney, muscle or any other tissue in a subject. One skilled in the art could also utilize the methods of the present invention to administer therapeutic agents to tissues in vivo or ex vivo according to standard methods. For example, prior to transplantation, a therapeutic agent and a facilitating agent can be administered to a tissue to be transplanted to reduce immune rejection of the tissue upon subsequent transplantation in a subject. For example, VEGF (vascular endothelial growth factor) and its homologs or HGF (hepatocyte growth factor) can be delivered with heparin prior to transplantation.

For either ex vivo or in vivo use, therapeutic agents and facilitating agents of this invention can be administered at any effective concentration. An effective concentration of a therapeutic agent is one that results in decreasing or increasing a particular pharmacological effect. An effective concentration of a facilitating agent is an amount that results in increasing the volume of distribution and/or the pharmacological activity of a therapeutic agent as compared to the volume of distribution and/or pharmacological activity of the therapeutic agent in the absence of the facilitating agent. One skilled in the art would know how to determine effective concentration according to methods known in the art, as well as provided herein. For example, for a particular tissue to be targeted, cells from the target tissue are biopsied and optimal dosages for delivery of the therapeutic agent and facilitating agent into that tissue to achieve the desired distribution volume and/or pharmacological activity of the therapeutic agent are determined in vitro, allowing for the optimization of the in vivo dosage of the respective agents, including concentration and time course of administration.

Dosages of the therapeutic agents and facilitating agents of this invention will depend upon the disease or condition to be treated, and the individual subject's status (e.g., species, weight, disease state, etc.) Dosages will also depend upon the agents being administered. Such dosages are known in the art or can be determined as described above. Furthermore, the dosage can be adjusted according to the typical dosage for the specific disease or condition to be treated. Often a single dose can be sufficient; however, the dose can be repeated if desirable. The dosage should not be so large as to cause adverse side effects. Generally, the dosage will vary with the age, condition, sex and extent of the disease in the patient and can be determined by one of skill in the art according to routine methods (see e.g., Remington's Pharmaceutical Sciences (33)). The dosage can also be adjusted by the individual physician in the event of any complication.

The therapeutic agent and/or the facilitating agent of this invention can typically include an effective amount of the respective agent in combination with a pharmaceutically acceptable carrier and, in addition, may include other medicinal agents, pharmaceutical agents, carriers, adjuvants, diluents, etc. By “pharmaceutically acceptable” is meant a material that is not biologically or otherwise undesirable, i.e., the material may be administered to an individual along with the selected agent without causing any undesirable biological effects or interacting in a deleterious manner with any of the other components of the pharmaceutical composition in which it is contained.

It is also contemplated that the methods of the present invention can be utilized to treat solid tumors, for example, by administering an antitumorigenic agent and a facilitating agent during localized delivery into the tumor, such as localized injection, CED, intracerebral delivery and/or intraventricular delivery. An effective combination of an antitumorigenic agent and a facilitating agent is that combination that results in partial or total killing, reduction in size, disappearance, inhibition of growth, inhibition of vascularization, inhibition of cellular proliferation, an induction in dormancy or an apparent induction of dormancy, and/or a decreased metastasis of a tumor or a tumor cell. These mechanisms of action are only exemplary of the ways an antitumorigenic protein can treat a tumor. The subjects to be treated by the methods of this invention can include subjects undergoing additional anti-tumor therapy, which can include patients undergoing surgery, chemotherapy, radiotherapy, immunotherapy or any combination thereof. Examples of chemotherapeutic agents include cisplatin, 5-fluorouracil and S-1. Immunotherapeutic methods can include administration of interleukin-2 and interferon-α.

The present invention further provides a method of treating a neurodegenerative disorder in a subject in need of such treatment, comprising administering to the subject a therapeutic agent and a facilitating agent, wherein the therapeutic agent and the facilitating agent are administered via localized delivery such as convection enhanced delivery, localized injection, intracerebral delivery and/or intraventricular delivery. Other means of localized delivery include catheterization of an artery in the brain in order to supply agents such as mannitol or other sugars that are capable of disrupting the blood-brain-barrier to allow delivery of therapeutic and facilitating agents.

The neurodegenerative disorders that can be treated by the methods of the present invention include, but are not limited to, Parkinson's disease, Huntington's disease, Alzheimer's disease, ALS (amylotrophic lateral sclerosis), PSP (progressive supranuclear palsy), MSA (multiple system atrophy), SCA (autosomal dominant spinocerebellar ataxia) and other cerebellar ataxias.

For example, in a method of this invention employing intraventricular delivery, a catheter can be implanted in the ventricle of a subject diagnosed with a neurodegenerative disorder such that the appropriate therapeutic agent and facilitating agent can be injected into the ventricle via the catheter. Dosages will depend upon the disease or condition to be treated, and the individual subject's condition. Dosages will also depend upon the material being administered. Such dosages are known in the art or can be determined as described above. Furthermore, the dosage can be adjusted according to the typical dosage for the specific disease or condition to be treated. Often a single dose can be sufficient; however, the dose can be repeated if desirable. The dosage should not be so large as to cause adverse side effects. Generally, the dosage will vary with the age, condition, sex and extent of the disease in the subject and can be determined by one of skill in the art. The dosage can also be adjusted by the individual physician in the event of any complication. For guidance on intraventricular delivery, see Kordower et al. “Clinicopathological Findings following Intraventricular Glial-Derived Neurotophic Factor Treatment in a Patient With Parkinson's Disease” Ann. Neur. 46: 419-424 (1999),″ which is hereby incorporated by this reference in its entirety.

The invention further provides a method of treating a lysosomal storage disorder in a subject in need of such treatment, comprising administering to the subject a therapeutic agent and a facilitating agent, wherein the therapeutic agent and the facilitating agent are administered via localized delivery, such as convection enhanced delivery, intracerebral injection or intraventricular delivery.

Examples of lysosomal storage disorders that can be treated by the methods of this invention include, but are not limited to, Gaucher disease, Krabbe disease, Fabry disease, Tay-Sachs disease, Niemann-Pick disease type A/B, Niemann-Pick disease type C, Farber disease, neuronal ceroid lipofuscinosis (infantile), neuronal ceroid lipofuscinosis (late infantile), Schindler disease, metachromatic leukodystrophy, Pompe disease and Sandhoff disease (32).

Further provided by this invention is a method of increasing dopamine metabolism in a tissue in a subject during localized delivery such as convection enhanced delivery, intracerebral injection or intraventricular delivery, comprising administering to the tissue in the subject a therapeutic agent and a facilitating agent, whereby the inclusion of the facilitating agent increases dopamine metabolism in the tissue.

As used herein, “increased dopamine metabolism” means that dopamine utilization is greater when a therapeutic agent is administered with a facilitating agent as compared to dopamine utilization when the therapeutic agent is administered in the absence of a facilitating agent. Methods of measuring dopamine metabolism are described in the Examples.

The following examples are set forth so as to provide those of ordinary skill in the art with a complete disclosure and description of how the methods claimed herein may be performed, and are intended to be purely exemplary of the invention and are not intended to limit the scope of what the inventors regard as their invention.

### EXAMPLE

CED, a recently developed approach for delivery of small and large molecules to targeted sites in solid tissues, utilizes bulk flow to deliver and distribute macromolecules to clinically significant volumes of tissue (3, 21) This technique of drug delivery offers significant advantages over diffusion: (i) improved V_{d}, (ii) a more uniform concentration of drug distributed at the targeted region, and (iii) delivery of all of a therapeutic agent to the target site (thus exposing only the target site to the therapeutic agent and retrieving maximum effect from a very small dose) (21). Therefore, CED can be very useful in the delivery of therapeutic factors such as trophic factors to the CNS, especially those with a narrow range of effective concentrations (E_{c}) (3, 21). This method of delivery bypasses the blood brain barrier and therefore is not subject to the limitations of systemic delivery of a therapeutic agent.

Parkinson's disease (PD) is caused by degeneration of dopaminergic neurons that innervate the striatum. Therefore, trophic factors within the GDNF family, with their ability to improve dopaminergic cell survival in vitro and partially protect dopaminergic cells from various models of Parkinsonian injury (17, 10, 14, 30), have raised hopes that the GDNF family of trophic factors can be used as therapeutic agents in the treatment of PD.

In the study described in this Example, CED was used to deliver GDNF, NTN and ART within the rat striatum. With NTN, high-volume infusions into the rat striatum also were performed at volumes more relevant to primate infusions to approximate infusion parameters. The V_{d} of GDNF, NTN and ART, when infused alone, was considerably less than expected, based on estimates of molecular weight. Co-infusion with heparin dramatically increased the V_{d} of GDNF and it's homologues, but did not increase the distribution of the nonspecifically bound protein, BSA. Furthermore, co-infusion with heparin had no adverse effect on the bioactivity of NTN in vivo and, more importantly, produced a significant increase in dopamine utilization.

This demonstrated the importance of blocking receptors in the distribution of therapeutic agents in vivo and offers a model for drug delivery in the treatment of neurodegenerative diseases.

Animal Preparation

Sixty-five Sprague-Dawley rats, each weighing 275-350 g, were used. All procedures were performed in accordance with the regulations of the Animal Care and Use Committee of the National Institute of Neurological Disorders and Stroke.

Surgery and Convection Procedure

The animals were anesthetized with ketamine (10 mg/kg) and xylazine (3 mg/Kg) via intraperitoneal injection and placed in a Kopf small animal stereotactic frame. A sagittal incision was made through the skin and a burr hole was placed into the skull with a twist drill. The cannula coordinates were: 0.5 mm anterior to bregma, 2.8 mm to the right of bregma, and 5 mm below the dura.

The infusion apparatus consisted of a hydraulic drive serially connected to a syringe pump. This depressed the plunger of one of two 250 μl Hamilton syringes connected by cannulas made from polyetheretherketone (PEEK) tubing (inner diameter 250 μm) and filled with distilled water. One of the 250 μl Hamilton syringes was secured to the stereotactic frame and was contiguous with an infusate-filled 10 or 50 μl Hamilton syringe. Since the system was non-compliant (zero dead space), the compression of the syringe pump of the infusion apparatus led to the measured release of infusate from the 10 or 50 μl Hamilton syringe in a uniform fashion. All infusions were performed with a 32 g cannula at 0.2 μl/min (6).

In the low volume experiment, five micrograms of GDNF, NTN, ART or BSA in 5 μl of vehicle with and without heparin (18 μg) was infused at 0.2 μl/min through a 32 g cannula. The vehicle was 4% mannitol in 10 mM HEPES. In the high volume experiment, 50 μg of NTN in 20 μl of vehicle with (n=6) and without (n=6) heparin (71 μg) was delivered into the striatum.

In the study analyzing dopamine metabolism following trophic factor administration, the animals were infused with 5 μg of NTN in 5 μl of vehicle with and without heparin (2.5 units) and sacrificed after 4 days. The vehicle was 4% mannitol in 10 mM HEPES. After completing infusion, the cannula was withdrawn at 1 mm/minute. Following the procedure, the animals used for immunohistochemical analysis were intracardially perfused with 4% paraformaldehyde. Brains were harvested and immersed overnight in 4% paraformaldehyde. The brains were cryoprotected through immersion in graded sucrose solution (20-30%) and frozen at 70° C. Animals to be used in HPLC studies were euthenized and the brains were harvested and fresh frozen at −70°.

Immunohistochemistry

The brains were cut into 40 μm serial coronal sections on a cryostat. Frozen sections were collected in a series in antifreeze solutions and stored at −70° C. Every 12^{th} section was stained for immunohistochemistry.

Sections were washed in phosphate buffered saline (PBS) and incubated in 3% H₂O₂ for 20 minutes to block endogenous peroxidase activity. After washing in PBS, the sections were incubated in blocking solution (10% normal horse serum and 0.1% Triton-X 100 in PBS) for 30 minutes, followed by incubation in the respective anti-GDNF, NTN, ART, BSA antibody solution (mouse monoclonal 1:1000) for 24 hours. The sections were then incubated for 1 hour in biotinylated anti-mouse IgG secondary antibody (Vector Labs, 1:300). Antibody binding was visualized with streptavidin horseradish peroxidase (Vector Labs, 1:300) and VIP chromogen (Vector Labs). Sections were then coverslipped and examined under light microscopy. V_{d} was measured using NIH image analysis software.

Image Analysis

The volume of distribution of BSA and the GDNF family of trophic factors was analyzed using a Macintosh-based image analysis system. Images of stained tissue slices were captured by a CCD camera using Adobe Photoshop software. Using NIH Image Analysis software, the area of distribution of infused protein in each tissue section was automatically determined using a threshold of 50% of the maximal stained optical density. The sum of the areas of infusion was used to determine the V_{d} in each striatum.

Biochemical studies

Animals were sacrificed 4 days after intrastriatal infusion of 5 μl vehicle with 18 μg of heparin, 5 μg of NTN in 5 μl of vehicle or 5 μg of NTN in 5 μl of vehicle with 18 μg of heparin. After decapitation, the brains were rapidly removed and chilled in ice for 4 minutes. They were sliced 1 mm anterior and 2 mm posterior to the injection site using a chilled plastic brain mold. For biochemical studies, frozen punches from the striatum were taken from the injected and uninjected sides for comparison. Tissue samples were collected, weighed, and homogenized in 500 μl of 0.1 M perchloric acid containing 1% ethanol and 0.02% disodium ethylenediamine tetraacetate (EDTA). The homogenates were centrifuged at 1900×g at 4° C. for 15 minutes. 10 to 100 μl of supernatant was used for catecholamine and indolamine analysis by HPLC and 30 μl was derivatized for amino acid analysis. Dopamine (DA), serotonin (5-HT) and their metabolites were measured by HPLC using Ultrasphere C-18 ion pair column, 5μ, 4.6 mm×25 cm (Beckman 235329): a Waters 717 plus autosampler, Waters 510 pump at 0.8 ml/min, and an amperometric electrochemical detector (EiCom CB-100) set at 0.78 V. The mobile phase contained 2-1 deionized water, 2.8 g L-heptanesulfonic acid sodium salt, 0.17 g EDTA, 20 ml triethylamine and 50 ml acetonitrile, pH was adjusted to 2.6 with 13 ml of 85% phosphoric acid. The results were recorded and analyzed with Waters Millenium 2010 Chromatography Manager software. Protein concentration in the tissue pellet was determined using the BCA Protein Assay Kit (Pierce #23225). The results are expressed as nM/hr/mg of protein.

Microdialysis

To investigate the in vivo synthesis of dopamine in response to L-DOPA, microdialysis was performed in 14 rats, 8-12 weeks after transfection. Rats were maintained under deep isoflurane anesthesia as described for surgical procedures. Microdialysis probes (BR-4, 4 mm exposed dialysis membrane with a recovery for DA of 23%, Bioanalytical Systems Inc., West Lafayette Ind., USA) were stereotactically inserted into the striatum at AP 0 mm, ML+/−3 mm and DV-6 mm (Paxinos). Both sides were inserted simultaneously. Artificial cerebrospinal fluid (aCSF; NaCl 145 mM, CaCl₂ 1.2 mM, KCl 2.7 mM, and MgCl₂ 1.0 mM, pH6.5) was pumped through the microdialysis probe at 2.0 μl/min using a microinjection pump (BAS). The dialysate was collected every 20 min (40 μL) into 250 μl microtubes containing 15 μl preservative (0.1 M perchloric acid with 0.02% EDTA and 1% ethanol), mixed, and frozen in dry ice until injected into an HPLC equipped with an electochemical detector (ECD). The HPLC-ECD was the same as previously described (Lammensdorf et al., 1999) except that the following column and mobile phase were used. The column was the Luna 150×2.0 mm, 5 μ, C18(2) (Phenomenex #OOF-4252-B0, Torrance, Calif., USA) held at 28.3° C. with a flow rate of 0.4 ml/min. The mobile phase consisted of 2.1 L HPLC grade water, 2.8 g 1-heptanesulfonic acid (# 0-3013, Fischer Scientific, Fairlawn, N.J., USA), 0.17 g EDTA (Fischer #S-311), 20 ml triethylamine (Fischer #0-4884), 50 ml acetonitrile (#015-4 Burdick & Jackson, Muskegon, Mich., USA), and the pH adjusted to 2.5 with 85% phosphoric acid (Fischer #A-260-500). L-DOPA methyl ester (50-100 mg/kg) pargyline (70 mg/kg) and benserazide (2.5 mg/kg, all from Sigma,) were disolved in sterile saline and administered i.p. Pargyline was administered at the beginning of the experiment to inhibit MAO oxidation of dopamine and prolong its half-life. Benserazide was given 40 min before L-DOPA administration to prevent peripheral decarboxylation. L-DOPA (50 mg/g) was administered to measure in vivo the activity of the transgenic AADC. Preliminary experiments were performed with different doses of L-DOPA and inhibitors. Omission of peripheral AADC inhibition (to prevent central inhibition that can occur as a result of the increase in BBB permeability associated to probe insertion) was not possible after pargyline administration as animals die shortly after L-DOPA administration as reported by others (Leff et al, 1998). KCl challenge was performed through the probe by switching to KCl-aCSF (NaCl was reduced to 36.9 mM, and KCl was increased to 110.8 MM) for 15 min. Samples were collected during 4-6 hours and at the end of the experiment animals were euthanized with an overdose of pentobarbital.

Sources of Supplies and Equipment

The stereotactic frame was purchased from Kopf instruments (Tujunga, Calif.), the syringe pump (model 22) was purchased from Harvard Apparatus (S. Natick, Mass.), and 10 μl, 50 μl and 250 μl Hamilton syringes were purchased from Thomson Instruments (Chantilly, Va.). ART, GDNF, NTN protein and antibodies to the GDNF family of trophic factors was generously provided by Genentech (San Francisco, Calif.). BSA and BSA antibody were purchased from Sigma-Aldrich (St.Louis, Mo.). Heparin (140 units per mg) was purchased from Elkins-Sinn (Cherry Hill, N.J.). The NIH Image 162 software program was developed by W. Rasb and and is available from the MIH (Bethesda, Md.). Statistical tests were performed using the software STATVIEW (SAS Institute) and a Macintosh G3.

Statistical Analysis

The experimental data were statistically analyzed by means of an unpaired student t-test or by a Fisher test as indicated. Results were provided as the mean +/−S.D or the mean +/−S.E. as indicated.

Low Volume Infusion of GDNF Family of Trophic Factors

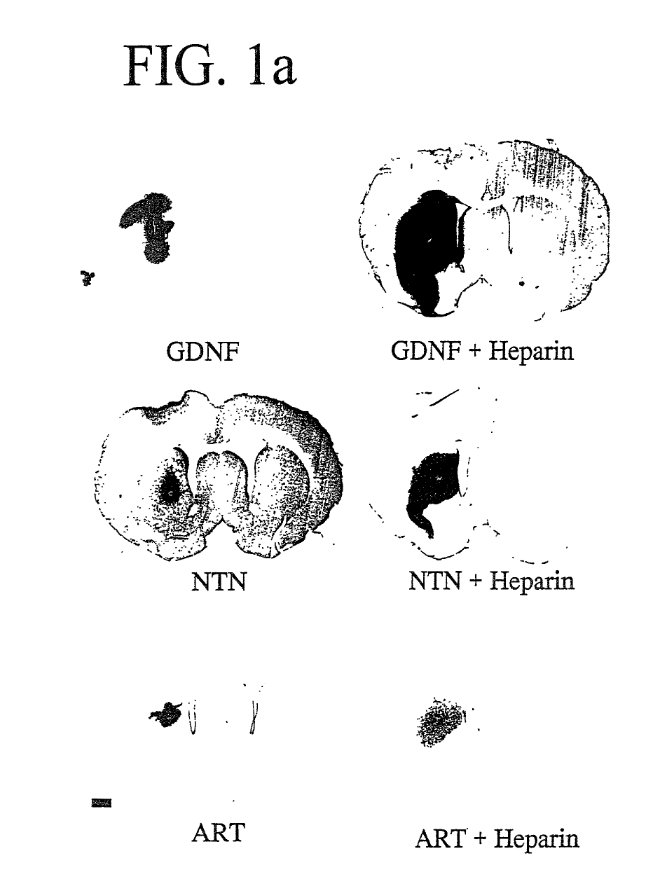

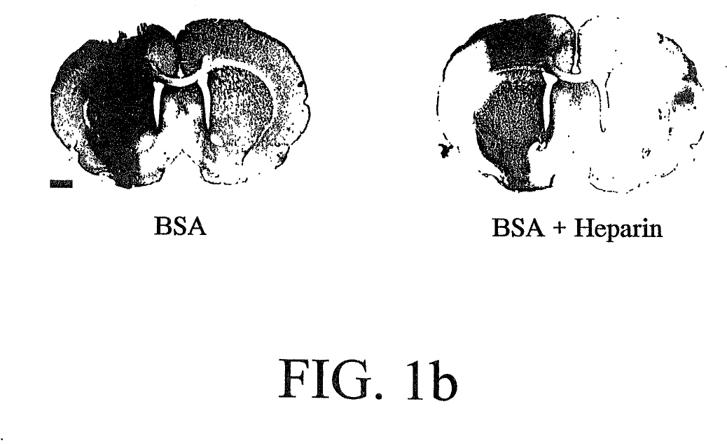

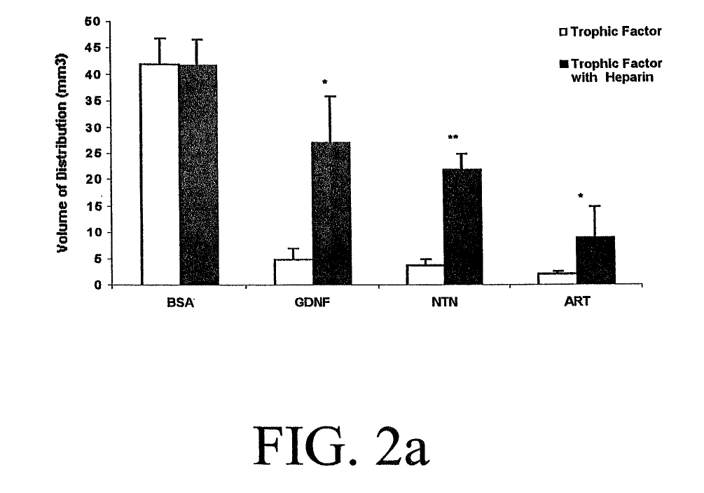

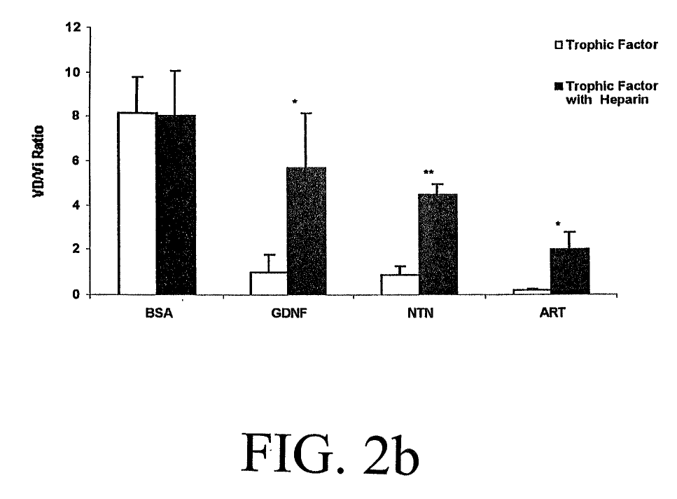

Heparin was co-infused into the striatum with BSA and with GDNF and GDNF-homologous trophic factors to evaluate the V_{d} of these substances in grey matter and the influence of heparin on the V_{d}. The V_{d} of low volume (5 μl) infusions of GDNF and GDNF-homologous trophic factors was significantly increased when co-infused with heparin (FIG. 1a), which was not noted with the non-specifically bound BSA (FIG. 1b). Heparin co-infusion dramatically increased the V_{d} of NTN in the striatum (P=0.0001). A similar effect of heparin co-infusion occurred for the other growth factors. In contrast, heparin co-infusion with BSA produced no change in V_{d} (FIG. 2a and 2b). Among the trophic factors in the GDNF family, NTN and GDNF had the greatest V_{d}, compared to ART. However, ART demonstrated the greatest increase in V_{d} when co-infused with heparin, with a greater than 10-fold increase. This compares favorably with the 5-fold increase and 7-fold increase in V_{d} in GDNF and NTN, respectively, when co-infused with heparin. GDNF and NTN had the highest V_{d}/Vᵢ ratio, 5.0 and 4.6, respectively, which was approximately half of the V_{d}/Vᵢ ratio noted with BSA.

High Volume Neurturin Infusions

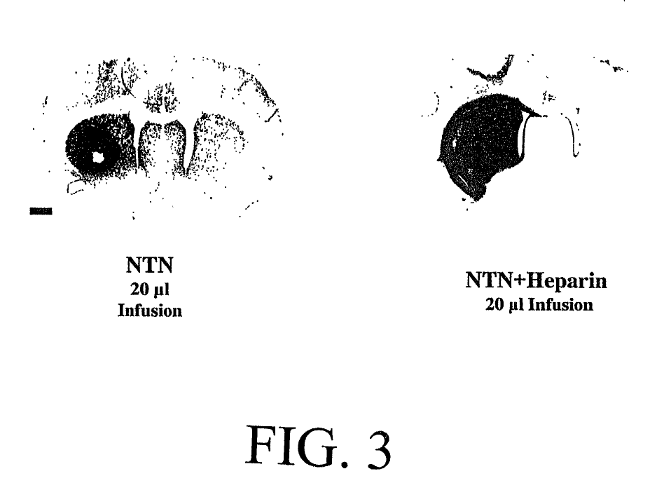

Studies were conducted to establish whether CED could be used to distribute trophic factors within the GDNF family over volumes that might be relevant to larger primate brains. High volume infusions (20 μl) of NTN with heparin were able to cover significant volumes of the rat brain (FIG. 3). At large volumes, heparin dramatically increased the V_{d} (from 7.7+/−1.1 to 32.2+/−3.1 mm³; P<0.0001). Maximum V_{d} attained following co-infusion with heparin was 37.3 mm³, compared to a maximum Vd of 11.0 mm³ in control animals (FIG. 3). At this infusion volume, in 4 out of 6 animals in which NTN was co-infused with heparin, the striatum was completely stained, as opposed to none of the subjects infused with NTN alone. There was no evidence of hemorrhage (H&E stain) in any of the specimens following the high volume infusions.

Biochemistry

GDNF and NTN increase dopamine utilization (13,18). Given that co-infusion with heparin increased the V_{d} of trophic factors within the GDNF ligand family, further experiments were done to determine whether the trophic factors retained biological activity in the presence of heparin. In this portion of the study, we examined whether heparin co-infusion was examined for the ability to block this activity by comparing the DOPAC/DA ratio at 4 days after infusion. The DOPAC/DA ratio in each subject was normalized against the uninjected left striatum. Animals which received vehicle with heparin experienced no change in the DOPAC/DA levels when compared to the uninjected (left) striatum. Both groups of rats which received NTN and NTN+heparin experienced a significant increase in the DOPAC/DA ratio (146+/−14.2 (P=0.0179) and 243+/−17.4 (P<0.0001), respectively). Moreover, co-infusion of heparin with NTN induced a significant increase in the DOPAC/DA ratio at 4 days when compared with the group that received NTN alone (P=0.0004).

All brains had well demarcated areas of antibody staining following immunohistochemistry. In all brains, there was no evidence of tissue damage, with the exception of the needle track and some non-specific drill-induced cortical lesions. There was no evidence of hemorrhage in any animal at 0 or 4 days after infusion.

This study demonstrates the importance of receptor binding in drug delivery. Heparin acts to increase the V_{d} of the GDNF family of trophic factors by blocking the binding to heperan sulfate proteoglycan in the ECM. This is congruent with in vitro studies in which heparin was shown to increase the ability of bFGF to diffuse in an agarose gel (9). The ability to deliver these trophic factors has important implications in the treatment of neurodegenerative disorders such as Parkinson's disease, as they have been demonstrated to play a key role in the development, maintenance, and repair of dopaminergic neuron (10, 12, 13, 30).

As research into the use of trophic factors for neurodegenerative disorders has progressed, promising results in vitro have not fully been reproduced in in vivo studies. With GDNF and BDNF, this is probably related to limited V_{d} of the trophic factor because of binding sites in the ECM and along the ependymal lining (15, 23). For the GDNF ligand family, co-infusion with heparin provides a mechanism to increase distribution of the factor within the target tissue without blocking its activity.

The increase of V_{d} with heparin co-infusion did not occur with BSA. V_{d} of BSA was significantly greater than that of GDNF and its homologues, despite that the molecular weight of BSA is more than twice that of the trophic factors. This emphasizes the importance of receptor binding in drug distribution as well. At the concentration of heparin that was used in the low volume studies, 0.5 units/μl, there was no evidence of gross or microscopic hemorrhage in any of the infused animals.

With the high volume infusions, this invention demonstrated that this method can be used to deliver trophic factors to greater volumes of tissue in proportion to higher volumes of infusate. In fact, in 4 out of 6 animals, the entire striatum was covered. GDNF's negligible benefit in recent clinical trials may be attributable to limited tissue distribution (15). Heparin co-infusion offers a way to overcome this limitation.

Throughout this application, various publications are referenced. The disclosures of these publications in their entireties are hereby incorporated by reference into this application in order to more fully describe the state of the art to which this invention pertains.

REFERENCES

1. Amalric, F., Bouche, G., Bonnet, H., Brethenou, P., Roman, A. M., Truchet, I., and Quarto, N. 1994. Fibroblast Growth Factor-2 (FGF-2) in the nucleus: translocation process and targets. Biochem. Pharmacol. 47: 111-115.

2. Baloh, R. H., Tansey, M. G., Lampe, P. A., Fahrner, T. J., Enomoto, H., Simburger, K. S., Leitner, M. L., Araki, T., Johnson, E. M. Jr. and Milbrandt J. 1998. Artemin, a novel member of the GDNF ligand family, supports peripheral and central neurons and signals through the GFR alpha3-RET receptor complex. Neuron 21: 1291-1302.

3. Bobo, R. H., Laske, D. W., Akbasak A., Morrison, P. F., Dedrick, R. L., Oldfield, E. H. 1994. Convection-enhanced delivery of macromolecules in the brain. Proc. Natl. Acad. Sci. USA 91:2076-2080.

4. Cabelli, R. J., Shelton, D. L., Segal, R. A. and Shatz, C. J. 1997. Blockade of endogenous ligands of trkB inhibits formation of ocular dominance columns. Neuron 19:63-76.

5. Croll, S. D. Chesnutt C. R., Rudge, J. D., Acheson A., Ryan, T. E., Siuciak, J. A., DiStefano, P. S., Wiegand S. J., Lindsay R. M. 1998. Co-infusion with a TrkB-Fc receptor body carrier enhances BDNF distribution in the adult rat brain. Exp. Neurol. 152: 20-33.

6. Chen, M. Y., Lonser, R. R., Morrison, P. F., Govemale, L. S. and Oldfield, E. H. 1999. Variables affecting convection-enhanced delivery to the striatum: a systematic examination of rate of infusion, cannula size, infusate concentration, and tissue-cannula sealing time. J. Neurosurg. 90: 315-320.

7. Emerich, D. F. Winn S. R., Hantraye, P. M., Peschanski, M., Chen E. Y., Chu, Y., McDermott, P., Baetge, E. E., Kordower, J. H. 1997. Protective effect of encapsulated cells producing neurotrophic factor CNTF in a monkey model of Huntington's disease. Nature 386: 395-399.

8. Figurov, A., Pozzo-Miller, L. D., Olafsson, P., Wang, T. and Lu, B. 1996. Regulation of synaptic responses to high-frequency stimulation and LTP by neurotrophins in the hippocampus. Nature 381: 706-709.

9. Flaumenhaft, R., Moscatelli D., and Rifkin D. B. 1990. Heparin and heparan sulfate increase the radius of diffusion and action of basic fibroblast growth factor. J. Cell Biol. 111: 1651-1659.

10. Gash, D. M. Zhang, Z., Ovadia, A., Cass, W. A., Yi, A., Simmerman, L., Russel, D., Martin, D., Lapchak, P. A., Collins, F., Hoffer, B. J., Gerhardt, G. A. 1996. Functional recovery in parkinsonian monkeys treated with GDNF. Nature 380: 252-255.

11. Gospodarowicz, D., Cheng, J., Lui, G. M., Baird, A. and Bohlent, P. 1984. Isolation of brain fibroblast growth factor by heparin-Sepharose affinity chromatography: identity with pituitary fibroblast growth factor. Proc. Nat.l Acad. Sci. USA 81: 6963-6967.

12. Hoffer, B. J. Hoffman, A., Bowenkamp, K., Huettl, P., Hudson, J., Martin, D., Lin, L. F., Gerhardt, G. A. 1994. Glial cell line-derived neurotrophic factor reverses toxin-induced injury to midbrain dopaminergic neurons in vivo. Neurosci. Lett. 182: 107-111.

13. Horger, B. A. Nishimura, M. C., Armanini, M. P., Wang, L. C., Poulsen, K. T., Rosenblad, C., Kirik, D., Moffat, B., Simmons, L., Johnson, E. Jr, Milbrandt, J., Rosenthal, A., Bjorklund, A., Vandlen, R. A., Hynes, M. A., Phillips, H. S. 1998. Neurturin exerts potent actions on survival and function of midbrain dopaminergic neurons. J. Neurosci. 18: 4929-4937.

14. Kearns, C. M. and Gash, D. M. 1995. GDNF protects nigral dopamine neurons against 6-hydroxydopamine in vivo. Brain. Res. 672: 104-111.

15. Kordower, J. H. Palfi, S., Chen, E. Y., Ma, S. Y., Sendera, T., Cochran, E. J., Cochran, E. J., Mufson, E. J., Penn, R., Goetz, C. G., Comella, C. D. 1999. Clinicopathological findings following intraventricular glial-derived neurotrophic factor treatment in a patient with Parkinson's disease. Ann. Neurol. 46: 419-424.

16. Kotzbauer, P. T., Lampe, P. A., Heuckeroth, R. O. Golden, J. P., Creedon, D. J., Johnson Jr. E. M. and Milbrandt J. 1996. Neurturin, a relative of glial cell line derived neurotrophic factor. Nature 384: 467-470.

17. Lieberman, D. M., Laske, D. W., Morrison, P. F., Bankiewicz, K. S. and Oldfield, E. H. 1995. Convection-enhanced distribution of large molecules in gray matter during interstitial drug infusion. J. Neurosurg. 82: 1021-1029.

18. Lin, L. F., Doherty D. H., Lile, J. D., Bektesh, S. and Collins, F. 1993. GDNF: a glial cell line derived neurotrophic factor for midbrain dopaminergic neurons. Science 260: 1130-1132.

19. Milbrandt, J., de Sauvage, F. J, Fahrner, T. J, Baloh, R. H., Leitner, M. L., Tansey, M. G., Lampe, P. A., Heuckeroth, R. O., Kotzbauer, P. T., Simburger, K. S., Golden J. P., Davies, J. A., Vejsada, R., Kato, A. C., Hynes, M., Sherman, D., Nishimura, M., Wang, L. C., Vandlen, R., Moffat, B., Klein, R. D., Poulsen, K., Gray, C., Garces, A., Johnson E. M. Jr., et al. 1998. Persephin; a novel neurotrophic factor related to GDNF and neurturin. Neuron 20: 245-253.

20. Milward, E. A. Papadopulos R., Fuller, S. J., Moir, R. D., Small, D., Beyreuther, K., Masters, C. L. 1992. The amyloid protein precursor of Alzheimer's disease is a mediator of the effects of nerve growth factor on neurite outgrowth. Neuron 9: 129-137.

21. Morrison, P. F., Laske, D. W., Bobo, H., Oldfield, E. H. and Dedrick, R. L. 1994. High-flow microinfusion: tissue penetration and pharmacodynamics. Am. J. Physiol. 266: R292-305.

22. Moscatelli, D. 1987. High and low affinity binding sites for basic fibroblast growth factor on cultured cells: absence of a role for low affinity binding in the stimulation of plasminogen activator production by bovine capillary endothelial cells. J. Cell Physiol. 131,: 123-130.

23. Mufson, E. J., Kroin, J. S., Liu, Y. T., Sobreviela, T., Penn, R. D., Miller, J. A., Kordower, J. H. 1996. Intrastriatal and intraventricular infusion of brain-derived neurotrophic factor in the cynomologous monkey: distribution, retrograde transport and co-localization with substantia nigra dopamine-containing neurons. Neuroscience 71: 179-191.

24. Poltorak, Z. Cohen, T., Sivan, R., Kandelis, Y., Spira, G., Vlodavsky, I., Keshet, E., Neufeld, G. 1997. VEGF145, a secreted vascular endothelial growth factor isoform that binds to extracellular matrix. J. Biol. Chem. 272: 7151-7158.

25. Ruppert, R., Hoffmann, E. and Sebald, W. 1996. Human bone morphogenetic protein 2 contains a heparin-binding site which modifies its biological activity. Eur. J. Biochem. 237: 295-302.

26. Saarma, M. and Sariola, H. 1999. Other neurotrophic factors: glial cell line-derived neurotrophic factor (GDNF). Microsc. Res. Tech. 45: 292-302.

27. Sauer, H., Rosenblad, C. and Bjorklund, A. 1995. Glial cell line-derived neurotrophic factor but not transforming growth factor beta 3 prevents delayed degeneration of nigral dopaminergic neurons following striatal 6-hydroxydopamine lesion. Proc. Natl. Acad. Sci. USA 92: 8935-8939.

28. Shing, Y. Folkman J., Sullivan, R., Buuterfield, C., Murray, J., Klagsbrun, M. 1984. Heparin affinity: purification of a tumor-derived capillary endothelial cell growth factor. Science 223: 1296-1299.

29. Strada, O., Hirsh, E. C., Javoy-Agid, F., Lehericy, S., Ruberg, M., Hauw, J. J., Agid, Y. 1992. Does loss of nerve growth factor receptors precede loss of cholinergic neurons in Alzheimer's disease? An autoradiographic study in the human striatum and basal forebrain. J. Neurosci. 12: 4766-4774.

30. Tomac, A. Lindqvist, E., Lin, L. F., Ogren, S. O., Young, D., Hoffer, B. J., Olson, L. 1995. Protection and repair of the nigrostriatal dopaminergic system by GDNF in vivo [see comments]. Nature 373: 335-339.

31. WuDunn, D. and Spear, P. G. 1989. Initial interaction of herpes simplex virus with cells is binding to heparan sulfate. J. Virol. 63: 52-58.

32. Meikle, P. J., Hopwood, J. J., Clague, A. E., Carey, W. F. 1999. Prevalence of Lysosomal Storage Disorders. JAMA 281: 249-254.

33. Martin EW: Remington's Pharmaceutical Sciences, latest ed., Mack Publishing Co., Easton, Pa.

What is claimed is:

1. A method of increasing the volume of distribution of a therapeutic agent in a tissue in a subject during localized delivery, comprising administering to the tissue in the subject a therapeutic agent and a facilitating agent, whereby the inclusion of the facilitating agent increases the volume of distribution of the therapeutic agent in the tissue.

2. The method of claim 1, wherein the localized delivery is selected from the group consisting of hypodermic injection, convection enhanced delivery, intracerebral injection and intraventricular injection.

3. The method of claim 1, wherein the therapeutic agent is a GDNF-ligand.

4. The method of claim 1, wherein the facilitating agent is heparin.

5. A method of increasing the pharmacological activity of a therapeutic agent in a tissue in a subject during localized delivery, comprising administering to the tissue in the subject a therapeutic agent and a facilitating agent, whereby the inclusion of the facilitating agent increases the pharmacological activity of the therapeutic agent in the tissue.

6. The method of claim 6, wherein the localized delivery is selected from the group consisting of convection enhanced delivery, hypodermic injection, intracerebral injection and intraventricular injection.

7. The method of claim 6, wherein the therapeutic agent is a GDNF-ligand.

8. The method of claim 6, wherein the facilitating agent is heparin.

9. A method of treating a neurodegenerative disorder in a subject in need of such treatment, comprising administering to the subject a therapeutic agent and a facilitating agent, wherein the therapeutic agent and the facilitating agent are administered via localized delivery.

10. The method of claim 9, wherein the localized delivery is selected from the group consisting of convection enhanced delivery, hypodermic injection, intracerebral injection and intraventricular injection.

11. The method of claim 9, wherein the GDNF-ligand is GDNF.

12. The method of claim 9, wherein the GDNF-ligand is NTN.

13. The method of claim 9, wherein the GDNF-ligand is ART.

14. The method of claim 9, wherein the neurodegenerative disorder is Parkinson's disease.

15. A method of increasing dopamine metabolism in a tissue in a subject during localized delivery, comprising administering to the tissue in the subject a therapeutic agent and a facilitating agent, whereby the inclusion of the facilitating agent increases dopamine metabolism in the tissue.

16. The method of claim 15, wherein the localized delivery is selected from the group consisting of: convection enhanced delivery, hypodermic injection, intracerebral injection and intraventricular injection.

17. The method of claim 15, wherein the GDNF-ligand is GDNF.

18. The method of claim 15, wherein the GDNF-ligand is NFN.

19. The method of claim 15, wherein the GDNF-ligand is ART.
